(19) United States
(12) Patent Application Publication
(10) Pub. No.: US 2002/0198252 A1
(43) Pub. Date: Dec. 26, 2002

(54) Process for the preparation of alkylamine substituted indoles

(76) Inventor: Joseph Payack, Somerset, NJ (US)

Correspondence Address: MERCK AND CO INC, P O BOX 2000, RAHWAY, NJ, 070650907

(21) Appl. No.: 10/153,357

(22) Filed: May 22, 2002

Related U.S. Application Data
(60) Provisional application No. 60/293,309, filed on May 24, 2001.

(51) Int. Cl. A61K 31/405; C07D 209/26

(52) U.S. Cl. 514/415; 548/452; 548/500

(57) ABSTRACT

The present invention relates to a process of synthesizing alkylamine-substituted indoles, which are useful as intermediates in the preparation of compounds known to useful in the treatment of cancer and other diseases by inhibiting, regulating and/or modulating tyrosine kinase signal transduction.

### BACKGROUND OF THE INVENTION

The present invention relates to a process of synthesizing alkylamine-substituted indoles, which are useful as intermediates in the preparation of compounds known to useful in the treatment of cancer and other diseases by inhibiting, regulating and/or modulating tyrosine kinase signal transduction.

Tyrosine kinases are a class of enzymes that catalyze the transfer of the terminal phosphate of adenosine triphosphate to tyrosine residues in protein substrates. Tyrosine kinases play critical roles in signal transduction for a number of cell functions by way of substrate phosphorylation. Though the exact mechanism of signal transduction is still unclear, tyrosine kinases have been shown to be important contributing factors in cell proliferation, carcinogenesis and cell differentiation.

Tyrosine kinases can be categorized as receptor type or non-receptor type. Receptor type tyrosine kinases have an extracellular, a transmembrane, and an intracellular portion, while non-receptor type tyrosine kinases are wholly intracellular.

The receptor-type tyrosine kinases are comprised of a large number of transmembrane receptors with diverse biological activity. In fact, about twenty different subfamilies of receptor-type tyrosine kinases have been identified. One tyrosine kinase subfamily, designated the HER subfamily, is comprised of EGFR, HER2, HER3, and HER4. Ligands of this subfamily of receptors include epithileal growth factor, TGF-α, amphiregulin, HB-EGF, betacellulin and heregulin. Another subfamily of these receptor-type tyrosine kinases is the insulin subfamily, which includes INS-R, IGF-IR, and IR-R. The PDGF subfamily includes the PDGF-α and β receptors, CSFIR, c-kit and FLK-II. Then there is the FLK family which is comprised of the kinase insert domain receptor (KDR), fetal liver kinase-1 (FLK-1), fetal liver kinase-4 (FLK-4) and the fms-like tyrosine kinase-1 (flt-1). The PDGF and FLK families are usually considered together due to the similarities of the two groups. For a detailed discussion of the receptor-type tyrosine kinases, see Plowman et al., DN&P 7(6):334-339, 1994, which is hereby incorporated by reference.

The non-receptor type of tyrosine kinases is also comprised of numerous subfamilies, including Src, Frk, Btk, Csk, Abl, Zap70, Fes/Fps, Fak, Jak, Ack, and LIMK. Each of these subfamilies is further sub-divided into varying receptors. For example, the Src subfamily is one of the largest and includes Src, Yes, Fyn, Lyn, Lck, Blk, Hck, Fgr, and Yrk. The Src subfamily of enzymes has been linked to oncogenesis. For a more detailed discussion of the non-receptor type of tyrosine kinases, see Bolen Oncogene, 8:2025-2031 (1993), which is hereby incorporated by reference.

Both receptor-type and non-receptor type tyrosine kinases are implicated in cellular signaling pathways leading to numerous pathogenic conditions, including cancer, psoriasis and hyperimmune responses.

Several receptor-type tyrosine kinases, and the growth factors that bind thereto, have been suggested to play a role in angiogenesis, although some may promote angiogenesis indirectly (Mustonen and Alitalo, J. Cell Biol. 129:895-898, 1995). One such receptor-type tyrosine kinase is fetal liver kinase 1 or FLK-1. The human analog of FLK-1 is the kinase insert domain-containing receptor KDR, which is also known as vascular endothelial cell growth factor receptor 2 or VEGFR-2, since it binds VEGF with high affinity. Finally, the murine version of this receptor has also been called NYK (Oelrichs et al., Oncogene 8(1):11-15, 1993). VEGF and KDR are a ligand-receptor pair that play an important role in the proliferation of vascular endothelial cells, and the formation and sprouting of blood vessels, termed vasculogenesis and angiogenesis, respectively.

Angiogenesis is characterized by excessive activity of vascular endothelial growth factor (VEGF). VEGF is actually comprised of a family of ligands (Klagsburn and D'Amore, Cytokine & Growth Factor Reviews 7:259-270, 1996). VEGF binds the high affinity membrane-spanning tyrosine kinase receptor KDR and the related fms-like tyrosine kinase-1, also known as Flt-1 or vascular endothelial cell growth factor receptor 1 (VEGFR-1). Cell culture and gene knockout experiments indicate that each receptor contributes to different aspects of angiogenesis. KDR mediates the mitogenic function of VEGF whereas Flt-1 appears to modulate non-mitogenic functions such as those associated with cellular adhesion. Inhibiting KDR thus modulates the level of mitogenic VEGF activity. In fact, tumor growth has been shown to be susceptible to the antiangiogenic effects of VEGF receptor antagonists. (Kim et al., Nature 362, pp. 841-844, 1993).

Solid tumors can therefore be treated by tyrosine kinase inhibitors since these tumors depend on angiogenesis for the formation of the blood vessels necessary to support their growth. These solid tumors include histiocytic lymphoma, cancers of the brain, genitourinary tract, lymphatic system, stomach, larynx and lung, including lung adenocarcinoma and small cell lung cancer. Additional examples include cancers in which overexpression or activation of Raf-activating oncogenes (e.g., K-ras, erb-B) is observed. Such cancers include pancreatic and breast carcinoma. Accordingly, inhibitors of these tyrosine kinases are useful for the prevention and treatment of proliferative diseases dependent on these enzymes.

The angiogenic activity of VEGF is not limited to tumors. VEGF accounts for most of the angiogenic activity produced in or near the retina in diabetic retinopathy. This vascular growth in the retina leads to visual degeneration culminating in blindness. Ocular VEGF mRNA and protein are elevated by conditions such as retinal vein occlusion in primates and decreased pO₂ levels in mice that lead to neovascularization. Intraocular injections of anti-VEGF monoclonal antibodies or VEGF receptor immunofusions inhibit ocular neovascularization in both primate and rodent models. Regardless of the cause of induction of VEGF in human diabetic retinopathy, inhibition of ocular VEGF is useful in treating the disease.

Expression of VEGF is also significantly increased in hypoxic regions of animal and human tumors adjacent to areas of necrosis. VEGF is also upregulated by the expression of the oncogenes ras, raf, src and mutant p53 (all of which are relevant to targeting cancer). Monoclonal anti-VEGF antibodies inhibit the growth of human tumors in nude mice. Although these same tumor cells continue to express VEGF in culture, the antibodies do not diminish their mitotic rate. Thus tumor-derived VEGF does not function as an autocrine mitogenic factor. Therefore, VEGF contributes to tumor growth in vivo by promoting angiogenesis through its paracrine vascular endothelial cell chemotactic and mitogenic activities. These monoclonal antibodies also inhibit the growth of typically less well vascularized human colon cancers in athymic mice and decrease the number of tumors arising from inoculated cells.

Viral expression of a VEGF-binding construct of Flk-1, Flt-1, the mouse KDR receptor homologue, truncated to eliminate the cytoplasmic tyrosine kinase domains but retaining a membrane anchor, virtually abolishes the growth of a transplantable glioblastoma in mice presumably by the dominant negative mechanism of heterodimer formation with membrane spanning endothelial cell VEGF receptors. Embryonic stem cells, which normally grow as solid tumors in nude mice, do not produce detectable tumors if both VEGF alleles are knocked out. Taken together, these data indicate the role of VEGF in the growth of solid tumors. Inhibition of KDR or Flt-1 is implicated in pathological angiogenesis, and these receptors are useful in the treatment of diseases in which angiogenesis is part of the overall pathology, e.g., inflammation, diabetic retinal vascularization, as well as various forms of cancer since tumor growth is known to be dependent on angiogenesis. (Weidner et al., N. Engl. J. Med., 324, pp. 1-8, 1991).

A number of compounds have been identified as inhibiting tyrosine kinase signal transduction, in particular as inhibitors of KDR. Several of these KDR inhibitors are characterized by an aminoalkylindole moiety, such as those illustrated in PCT Publ. WO 01/29025.

Accordingly, a practical, convergent synthesis of suitably substituted aminoalkylindole is desirable and is an object of this invention.

### SUMMARY OF THE INVENTION

The present invention relates to methods of preparing compounds that are synthetic intermediates of pharmaceutical compounds that inhibit, modulate and/or regulate signal transduction of both receptor-type and non-receptor type tyrosine kinases, in particular compounds that inhibit KDR. In particular the instant invention is directed to the synthesis of the intermediate compound of the formula I:

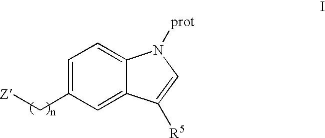

### DETAILED DESCRIPTION OF THE INVENTION

The instant invention is directed to a process for the preparation of the compound of Formula I:

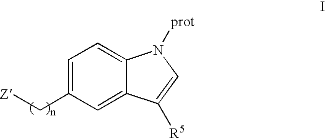

or a salt or optical isomer thereof;

wherein,

| | |
|---|---|
| a is | 0 or 1; |
| b is | 0 or 1; |
| m is | 0, 1, or 2; |
| n is | 1 to 10; |

Z′ is —NR⁷R⁸ or a protected precursor to —NR⁷R⁸;

R⁵ is selected from:

1) H,

2) (C═O)ₐO_{b}C₁-C₁₀ alkyl,

3) (C═O)ₐO_{b}aryl,

4) (C═O)ₐO_{b}C₂-C₁₀ alkenyl,

5) (C═O)ₐO_{b}C₂-C₁₀ alkynyl,

6) CO₂H,

7) halo,

8) OH,

9) O_{b}C₁-C₆ perfluoroalkyl,

10) (C═O)ₐNR⁷R⁸,

11) CN,

12) (C═O)ₐO_{b}C₃-C₈ cycloalkyl, and

13) (C═O)ₐO_{b}heterocyclyl,

said alkyl, aryl, alkenyl, alkynyl, cycloalkyl, and heterocyclyl is optionally substituted with one or more substituents selected from R⁶;

R⁶ is:

1) (C═O)ₐO_{b}C₁-C₁₀ alkyl,

2) (C═O)ₐO_{b}aryl,

3) C₂-C₁₀ alkenyl,

4) C₂-C₁₀ alkynyl,

5) (C═O)ₐO_{b}heterocyclyl,

6) CO₂H,

7) halo,

8) CN,

9) OH,

10) O_{b}C₁-C₆ perfluoroalkyl,

11) Oₐ(C═O)_{b}NR⁷R⁸,

12) oxo,

13) CHO,

14) (N═O)R⁷R⁸, or

15) (C═O)ₐO_{b}C₃-C₈ cycloalkyl,

said alkyl, aryl, alkenyl, alkynyl, heterocyclyl, and cycloalkyl optionally substituted with one or more substituents selected from R^{6a};

R^{6a} is selected from:

1) (C═O)ᵣOₛ(C₁-C₁₀)alkyl, wherein r and s are independently 0 or 1,

2) Oᵣ(C₁-C₃)perfluoroalkyl, wherein r is 0 or 1,

3) (C₀-C₆)alkylene-S(O)ₘR^{a}, wherein m is 0, 1, or 2,

4) oxo,

5) OH,

6) halo,

7) CN,

8) (C₂-C₁₀)alkenyl,

9) (C₂-C₁₀)alkynyl,

10) (C₃-C₆)cycloalkyl,

11) (C₀-C₆)alkylene-aryl,

12) (C₀-C₆)alkylene-heterocyclyl,

13) (C₀-C₆)alkylene-N(R^{b})₂,

14) C(O)R^{a},

15) (C₀-C₆)alkylene-CO₂R^{a},

16) C(O)H, and

17) (C₀-C₆)alkylene-CO₂H,

said alkyl, alkenyl, alkynyl, cycloalkyl, aryl, and heterocyclyl is optionally substituted with up to three substituents selected from R^{b}, OH, (C₁-C₆)alkoxy, halogen, CO₂H, CN, O(C═O)C₁-C₆ alkyl, oxo, and N(R^{b})₂;

R⁷ and R⁸ are independently selected from:

1) H,

2) (C═O)O_{b}C₁-C₁₀ alkyl,

3) (C═O)O_{b}C₃-C₈ cycloalkyl,

4) (C═O)O_{b}aryl,

5) (C═O)O_{b}heterocyclyl,

6) C₁-C₁₀ alkyl,

7) aryl,

8) C₂-C₁₀ alkenyl,

9) C₂-C₁₀ alkynyl,

10) heterocyclyl,

11) C₃-C₈ cycloalkyl,

12) SO₂R^{a}, and

13) (C═O)NR^{b}₂,

said alkyl, cycloalkyl, aryl, heterocylyl, alkenyl, and alkynyl is optionally substituted with one or more substituents selected from R6^{a}, or

R⁷ and R⁸ can be taken together with the nitrogen to which they are attached to form a monocyclic or bicyclic heterocycle with 5-7 members in each ring and optionally containing, in addition to the nitrogen, one or two additional heteroatoms selected from N, O and S, said monocylcic or bicyclic heterocycle optionally substituted with one or more substituents selected from R^{6a};

R^{a} is (C₁-C₆)alkyl, (C₃-C₆)cycloalkyl, aryl, or heterocyclyl;

R^{b} is H, (C₁-C₆)alkyl, aryl, heterocyclyl, (C₃-C₆)cycloalkyl, (C═O)OC₁-C₆ alkyl, (C═O)C₁-C₆ alkyl or S(O)₂R^{a}; and

prot is a nitrogen protecting group;

which comprises the steps of

a) reacting the compound of the formula II:

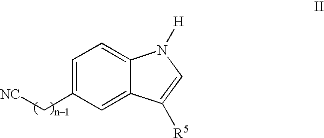

wherein n and R⁵ are defined hereinabove,

with a nitrogen protecting group reagent to provide a compound of the formula III:

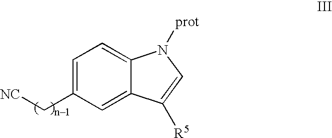

wherein n and R⁵ are defined hereinabove;

b) reacting the compound of the formula III with a cyano reducing agent to provide a compound of the formula IV:

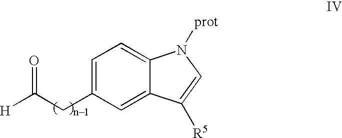

wherein n and R⁵ are defined hereinabove; and

c) reacting the intermediate of the formula IV with a suitably substituted amine HZ′, wherein Z′ is as defined hereinabove, in the presence of a reducing agent to provide the compound of the formula I.

In an embodiment the invention is directed to a process wherein the amine HZ′ utilized in step c) is selected from:

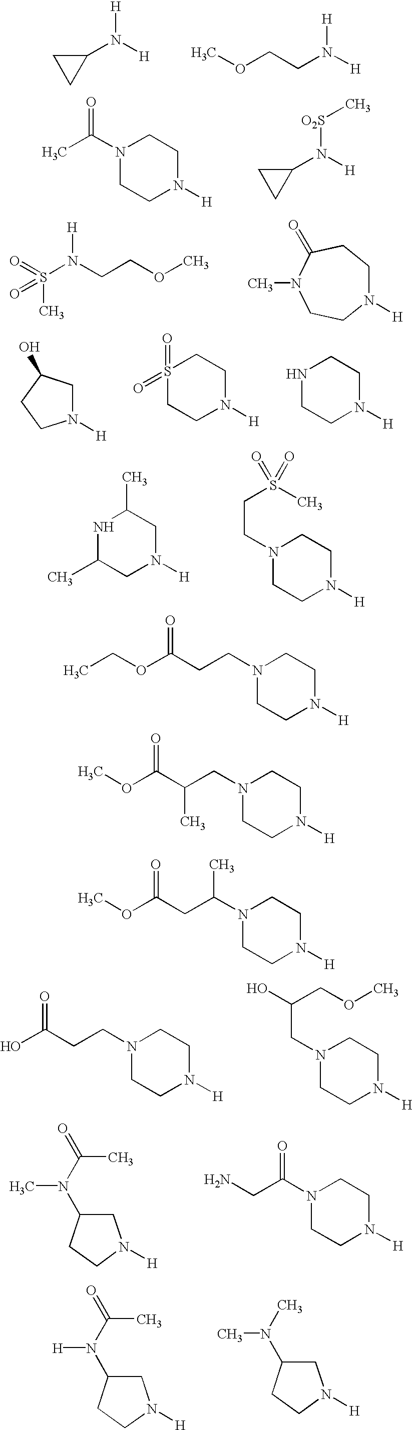

or a protected precusor to one of the amines above or a salt thereof.

In a preferred embodiment of the instant invention is directed to a process for the preparation of the compound of Formula Ia:

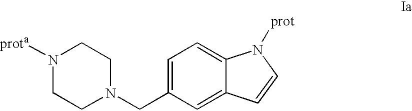

or a salt thereof;

wherein, prot and prot^{a} are independently selected from a nitrogen protecting group;

which comprises the steps of

c) reacting the compound of the formula IIa:

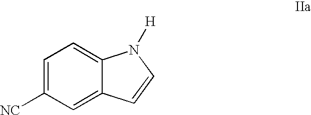

with a nitrogen protecting group reagent to provide a compound of the formula IIIa:

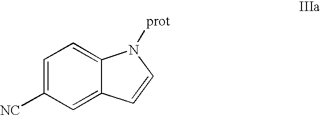

wherein prot is defined hereinabove;

d) reacting the compound of the formula IIIa with a cyano reducing agent to provide a compound of the formula IVa:

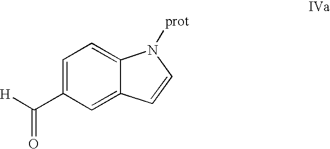

wherein prot is defined hereinabove; and

c) reacting the intermediate of the formula IVa with the amine

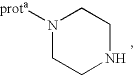

wherein prot^{a} is as defined hereinabove in the presence of a reducing agent to provide the compound of the formula Ia.

In a further preferred embodiment of the instant invention is directed to a process for the preparation of the compound of Formula 1-4:

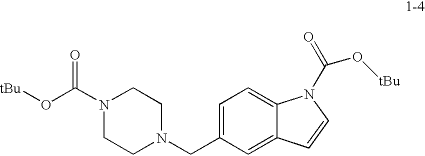

or a salt thereof;

which comprises the steps of

a) reacting the compound of the formula IIa:

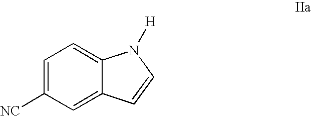

with a t-butoxycarbonyl anhydride to provide a compound of the formula IIIb:

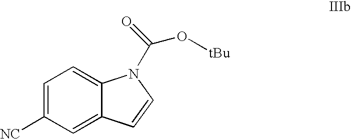

b) reacting the compound of the formula IIIb with a cyano reducing agent to provide a compound of the formula 1-2:

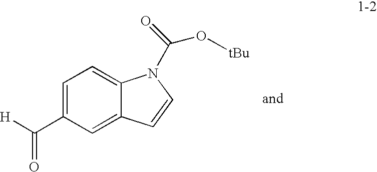

c) reacting the intermediate of the formula 1-2 with N-t-butoxycarbonylpiperidine in the presence of a reducing agent to provide the compound of the formula 1-4.

These and other aspects of the invention will be apparent from the teachings contained herein.

“Tyrosine kinase-dependent diseases or conditions” refers to pathologic conditions that depend on the activity of one or more tyrosine kinases. Tyrosine kinases either directly or indirectly participate in the signal transduction pathways of a variety of cellular activities including proliferation, adhesion and migration, and differentiation. Diseases associated with tyrosine kinase activities include the proliferation of tumor cells, the pathologic neovascularization that supports solid tumor growth, ocular neovascularization (diabetic retinopathy, age-related macular degeneration, and the like) and inflammation (psoriasis, rheumatoid arthritis, and the like).

The compounds of the present invention may have asymmetric centers, chiral axes, and chiral planes (as described in: E. L. Eliel and S. H. Wilen, Stereo-chemistry of Carbon Compounds, John Wiley & Sons, New York, 1994, pages 1119-1190), and occur as racemates, racemic mixtures, and as individual diastereomers, with all possible isomers and mixtures thereof, including optical isomers, being included in the present invention. In addition, the compounds disclosed herein may exist as tautomers and both tautomeric forms are intended to be encompassed by the scope of the invention, even though only one tautomeric structure is depicted.

When any variable (e.g. R⁶, R^{6a}, etc.) occurs more than one time in any constituent, its definition on each occurrence is independent at every other occurrence. Also, combinations of substituents and variables are permissible only if such combinations result in stable compounds. Lines drawn into the ring systems from substituents indicate that the indicated bond may be attached to any of the substitutable ring atoms. If the ring system is polycyclic, it is intended that the bond be attached to any of the suitable carbon atoms on the proximal ring only.

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results. The phrase “optionally substituted with one or more substituents” should be taken to be equivalent to the phrase “optionally substituted with at least one substituent” and in such cases the preferred embodiment will have from zero to three substituents.

As used herein the term “nitrogen protecting group” is intended to include moieties that may replace a hydrogen atom on a basic amine nitrogen which thereby renders that amine nitrogen less nucleophillic in reactivity. Such “nitrogen protecting groups” include groups that form a carbamate with the nitrogen, such as but not limited to fluorenylmethoxycarbonyl (FMOC), benzyloxycarbonyl (CBZ), t-butyloxycarbonyl (Boc) and the like. Preferably, both “prot” and “prot^{a}” are Boc.

As used herein the term “nitrogen protecting group reagent” is intended to include reagents that may react with a basic amine nitrogen in order to incorporate a nitrogen protecting group on that amine nitrogen. Such “nitrogen protecting groups reagents” include fluorenylmethoxycarbonyl chloride (FMOC-Cl), benzyloxycarbonyl chloride (CBZ-Cl), t-butyloxycarbonyl chloride (Boc-Cl), di-t-butyldicarbonate ((Boc)₂O) and the like. Preferably, the “nitrogen protecting group reagent” is (Boc)₂O.

As used herein the term “cyano reducing agent” is intended to include an aluminum hydride reducing agent, such as dilsobutylaluminum hydride (DiBAL-H), lithium aluminum hydride (LiAlH₄), sodium aluminum hydride (NaAlH₄), (LiAlH(OEt)₃), and the like. Preferably, the “cyano reducing agent” is DiBAL-H.

As used herein, the term “reducing agent” is intended to include reagents useful in a reductive amination. Such “reducing agents” include hydrogenation in the presence of a catalyst (such as Pd/C, PtO₂, and the like), sodium borohydride, sodium cyanoborohydride, BH₃/pyridine, sodium triacetoxyborohydride and the like.

As used herein, “alkyl” is intended to include both branched and unbranched, cyclic and acyclic saturated aliphatic hydrocarbon groups having the specified number of carbon atoms. For example, C₁-C₁₀, as in “C₁-C₁₀ alkyl” is defined to include groups having 1, 2, 3, 4, 5, 6, 7, 8, 9 or 10 carbons in a linear or branched arrangement and may be cyclic or acyclic. For example, “C₁-C₁₀ alkyl” specifically includes methyl, ethyl, n-propyl, i-propyl, n-butyl, t-butyl, i-butyl, pentyl, hexyl, heptyl, octyl, nonyl, decyl, cyclopropyl, methyl-cyclopropyl, 2,2-dimethyl-cyclobutyl, 2-ethyl-cyclopentyl, cyclohexyl, and so on. In some instances, definitions may appear for the same variable reciting both alkyl and cycloalkyl when a different number of carbons is intended for the respective substituents. The use of both terms in one definition should not be interpreted to mean in another definition that “alkyl” does not encompass “cycloalkyl” when only “alkyl” is used.

“Alkoxy” represents an alkyl group of indicated number of carbon atoms as defined above attached through an oxygen bridge.

If no number of carbon atoms is specified, the term “alkenyl” refers to a non-aromatic hydrocarbon radical, which may be branched or unbranched and cyclic or acyclic, containing from 2 to 10 carbon atoms and at least one carbon to carbon double bond. Preferably one carbon to carbon double bond is present, and up to four non-aromatic carbon-carbon double bonds may be present. Thus, “C₂-C₆ alkenyl” means an alkenyl radical having from 2 to 6 carbon atoms. Alkenyl groups include ethenyl, propenyl, butenyl, 2-methylbutenyl, cyclohexenyl, methylenylcyclohexenyl, and so on.

The term “alkynyl” refers to a hydrocarbon radical, which may be branched or unbranched and cyclic or acyclic, containing from 2 to 10 carbon atoms and at least one carbon to carbon triple bond. Up to three carbon-carbon triple bonds may be present. Thus, “C₂-C₆ alkynyl” means an alkynyl radical having from 2 to 6 carbon atoms. Alkynyl groups include ethynyl, propynyl, butynyl, 3-methylbutynyl and so on.

In certain instances, substituents may be defined with a range of carbons that includes zero, such as (C₀-C₆)alkylene-aryl. If aryl is taken to be phenyl, this definition would include phenyl itself as well as —CH₂Ph, —CH₂CH₂Ph, CH(CH₃)CH₂CH(CH₃)Ph, and so on.

As used herein, “aryl” is intended to mean phenyl and substituted phenyl, including moieties with a fused benzo group. Examples of such aryl elements include phenyl, naphthyl, tetrahydronaphthyl, indanyl, biphenyl, phenanthryl, anthryl or acenaphthyl. In cases where the aryl substituent is bicyclic, it is understood that attachment is via the phenyl ring. Unless otherwise indicated, “aryl” includes phenyls substituted with one or more substituents.

The term heteroaryl, as used herein, represents a stable monocyclic or bicyclic ring of up to 7 atoms in each ring, wherein at least one ring is aromatic and contains from 1 to 4 heteroatoms selected from the group consisting of O, N and S. Heteroaryl groups within the scope of this definition include but are not limited to: acridinyl, carbazolyl, cinnolinyl, quinoxalinyl, pyrrazolyl, indolyl, benzotriazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetrahydroquinoline. As with the definition of heterocycle below, “heteroaryl” is also understood to include the N-oxide derivative of any nitrogen-containing heteroaryl. In cases where the heteroaryl substituent is bicyclic and one ring is non-aromatic or contains no heteroatoms, it is understood that attachment is via the aromatic ring or via the heteroatom containing ring, respectively.

As appreciated by those of skill in the art, “halo” or “halogen” as used herein is intended to include chloro, fluoro, bromo and iodo.

The term “heterocycle” or “heterocyclyl” as used herein is intended to mean a 5- to 10-membered aromatic or nonaromatic heterocycle containing from 1 to 4 heteroatoms selected from the group consisting of O, N and S, and includes bicyclic groups. “Heterocyclyl” therefore includes the above mentioned heteroaryls, as well as dihydro and tetrathydro analogs thereof. Further examples of “heterocyclyl” include, but are not limited to the following: benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrahydropyranyl, tetrazolyl, tetrazolopyridyl, thiadiazolyl, thiazolyl, thienyl, triazolyl, azetidinyl, aziridinyl, 1,4-dioxanyl, hexahydroazepinyl, piperazinyl, piperidinyl, pyrrolidinyl, morpholinyl, thiomorpholinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, and tetrahydrothienyl, and N-oxides thereof. Attachment of a heterocyclyl substituent can occur via a carbon atom or via a heteroatom.

The alkyl, alkenyl, alkynyl, cycloalkyl, aryl, heteroaryl and heterocyclyl substituents may be unsubstituted or unsubstituted, unless specifically defined otherwise. For example, a (C₁-C₆)alkyl may be substituted with one, two or three substituents selected from OH, oxo, halogen, alkoxy, dialkylamino, or heterocyclyl, such as morpholinyl, piperidinyl, and so on. In this case, if one substituent is oxo and the other is OH, the following are included in the definition:

—(C═O)CH₂CH(OH)CH₃, —(C═O)OH, —CH₂(OH)CH₂CH(O), and so on.

In certain instances, R⁷ and R⁸ are defined such that they can be taken together with the nitrogen to which they are attached to form a monocyclic or bicyclic heterocycle with 5-7 members in each ring and optionally containing, in addition to the nitrogen, one or two additional heteroatoms selected from N, O and S, said heterocycle optionally substituted with one or more substituents selected from R^{6a}. Examples of the heterocycles that can thus be formed include, but are not limited to the following, keeping in mind that the heterocycle is optionally substituted with one or more substituents chosen from R^{6a}:

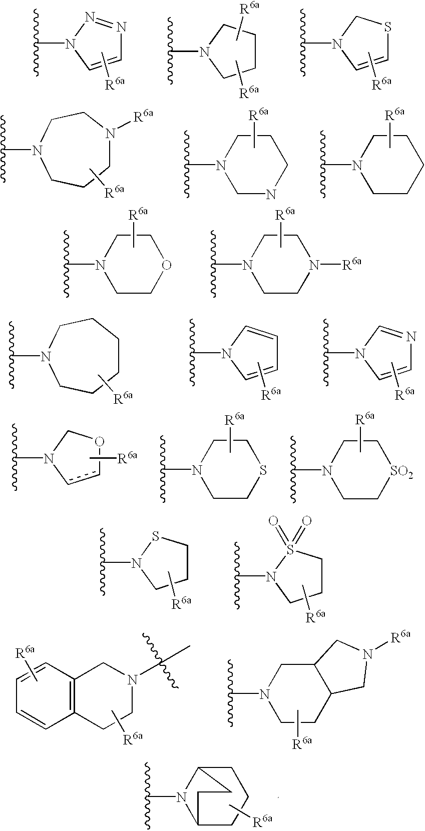

Preferably, n is 1.

Preferably, R⁵ is hydrogen.

The salts of compounds utilized in the instant processes include the conventional salts of the compounds, e.g., inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, palmoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, trifluoroacetic and the like.

With respect to compounds which contain an acid moiety, a salt may take the form, for example, —COOM, where M is a negative charge, which is balanced by a counterion, e.g., an alkali metal cation such as sodium or potassium. Other pharmaceutically acceptable counterions may be calcium, magnesium, zinc, ammonium, or alkylammonium cations such as tetramethylammonium, tetrabutylammonium, choline, triethylhydroammonium, meglumine, triethanolhydroammonium and the like.

The use of the process of the instant invention to prepare KDR inhibitors (such as those described in PCT Publ. WO 01/29025) is illustrated in the following schemes, in addition to other standard manipulations that are known in the literature or exemplified in the experimental procedures. These schemes, therefore, are not limited by the compounds listed or by any particular substituents employed for illustrative purposes. Substituent numbering as shown in the schemes does not necessarily correlate to that used in the claims. For example, the substitutent —NR^{7′}R^{8′} corresponds to Z′ of formula I.

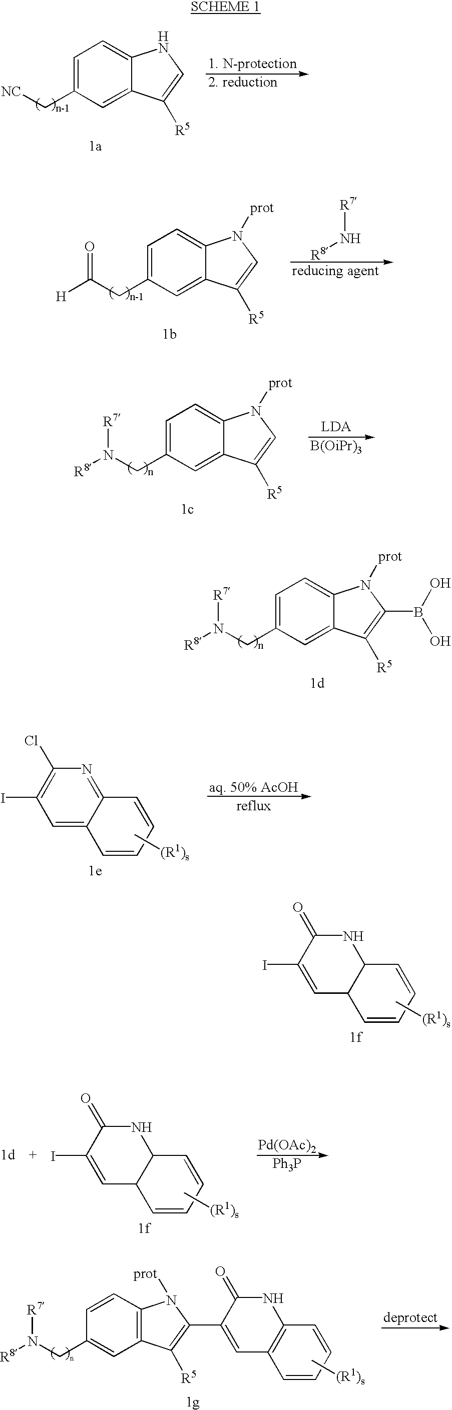

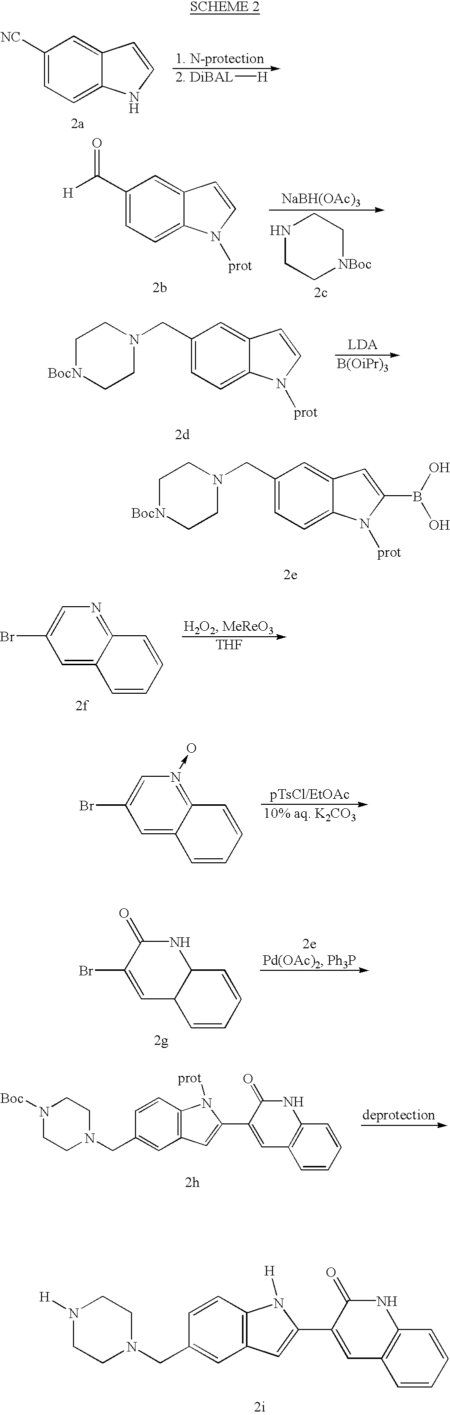

### EXAMPLES

Examples provided are intended to assist in a further understanding of the invention. Particular materials employed, species and conditions are intended to be illustrative of the invention and not limiting of the reasonable scope thereof.

### Example 1

### Tert-butyl 5-{[4-tert-(butoxycarbonyl)piperazin-1-yl]methyl}-1H-indole-1-carboxylate 1-4

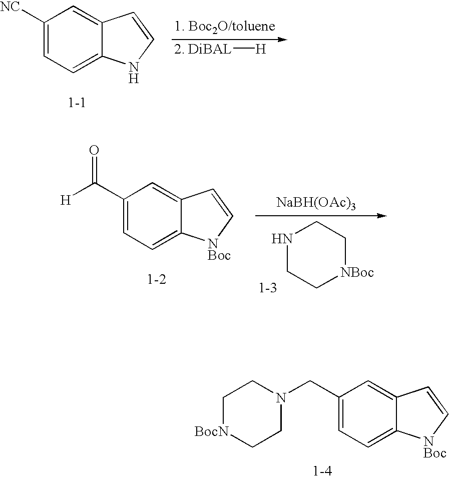

To a 50 L round bottomed flask was added toluene (8 L), 5-cyanoindole 1-1 (2 Kg, 1 eq.), and DMAP (17 g, 0.01 eq.). Boc₂O (3.15 Kg, 1.03 eq) was then added slowly as a solution in toluene (2 L), maintaining a temperature of about 20° C. to about 30° C. THF (8 L) was then added as a flush. After 30 minutes, the mixture was assayed using the HPLC assay described below and then cooled to a temperature of about 15° C. to about 18° C. DiBAL (21.5 L; 1.5 M in toluene; 2.3 eq.) was added over 3 hours, maintaining the temperature at about 15° C. to about 18° C. The solution was aged at room temperature for one hour to overnight, and then assayed by HPLC. Additional DiBAL (˜1 L) may be added to bring the assay of Boc-cyanoindole below 1 mol %.

The DiBAL reaction mixture was charged into half of an aqueous solution of NaHSO₄ (20 Kg) in water (60 L) while maintaining the temperature at about 35° C. to about 45° C. The rate of addition was governed by the ability to maintain the temperature at about 35° C. to about 45° C., and control the amount of gas evolution.

The aqueous phase was cut at about 35° C. to about 45° C. and the remaining bisulfate solution was charged to the organic phase. After a 15 minutes at 35° C. to about 45° C., the aqueous phase was cut and the organic phase was washed with water (8 L) and brine (8 L) before being transferred to carboys containing about 5 to about 10 Kg of Na₂SO₄ to remove second phase water. A small amount of red oil, residual over-reduced byproduct, appeared at the interface of the aqueous cuts, and was cut forward with the aqueous.

A 100 L extractor was washed with water and dried via THF boil-out, then the organic phase was recharged though a 10 micron line filter, followed by a toluene rinse (4 L). Boc-piperazine 1-3 (2.61 Kg, 1 eq) was added, then sodium triacetoxyborohydride (3.86 Kg, 1.3 eq) was added in portions while maintaining the temperature from about 23° C. to about 27° C. This addition was moderately exothermic. The mixture was aged for 1.5 hours, assayed and then quenched by adding 2.5 v/v % acetic acid in water (20 L). The total volume after quenching was about 80 L.

The organic phase was washed with water (20 L), the aqueous phase was cut and the organic phase solvent was switched to MeOH via in vacuo batch concentration in the 50 L round bottom to a target volume of 25 L. The batch was warmed to a temperature of about 30° C. to about 35° C. and seeded. After a good seed bed had formed, 60/40 water/methanol (20 L) was added over 1 hour and the batch chilled to about 5° C. and aged for 1 hour. The product was isolated via filtration, washed (3 L, 70:30 MeOH/water) and dried via a nitrogen purge. About 5 Kg (85%) of tert-butyl 5-{[4-tert-(butoxycarbonyl) piperazin-1-yl]methyl}-1H-indole-1-carboxylate 1-4 was obtained as a white solid.

### Example 2

Preparation of Boronic Acid Intermediate 2-1

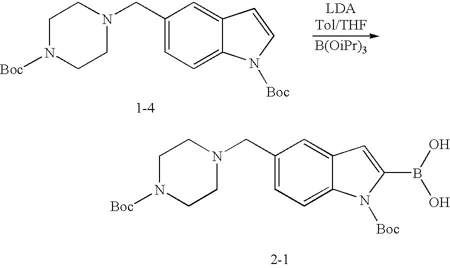

A mixture of 1-4 (2780 g; 6.69 mol), 11.1 L of toluene and 2.8 L of THF (tetrahydrofuran) was cooled to −78° C. 5.4 L (10.7 mol) of 2M LDA (lithium diisopropylamide) was then added slowly so as to keep the temperature below about −70° C. The reaction mixture was then aged for two hours.

4.6 L (19.9 mol) of triisopropylborate was added slowly while maintaining the temperature below about −70° C. The reaction is done when the remaining amount of 1-4 is two percent or less. Additional LDA may be added if necessary to drive the reaction to completion. After 30 minutes, the reaction was warmed to about 0° C. with an ice bath. The reaction was then quenched with 12 L of 2N HCl (24.1 mol) and the pH adjusted to about 7. The ice bath was removed and the biphasic solution was stirred for about 30 minutes to ensure that everything was in solution. The layers were then separated and the organic layer was used in the next reaction without further purification.

### Example 3

Preparation of Quinindole Intermediate 3-2

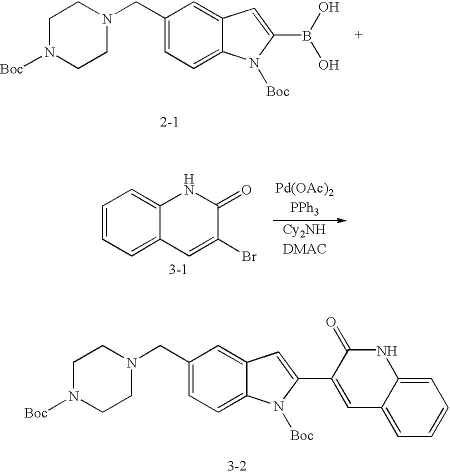

In a 50 L round bottom flask was combined the 3-bromoquinolin-2-one (1 kg, 4.46 moles), palladium acetate (50.1 g, 0.223 moles), PPh₃ (117 g, 0.446 moles), dicyclohexylamine (2.7 L, 13.4 moles) and dimethylacetamide (DMAC) (10 L). The solution was degassed two times and purged with nitrogen each time. The reaction mixture was heated to 60° C. At 60° C. the boronic acid (prepared as described in Example 2) (3.073 kg, 6.69 moles) was then added (this solution is not degassed) as a solution over a two hours period. The reaction was then aged overnight.

The reaction is assayed by HPLC. The reaction is done after the disappearance of either quinolinone or boronic acid. The ratio of desired product to undesired should be 3.5:1 or better.

Darco KB (125 g; 5 wt % of theory yield) was added to the reaction mixture. The mixture was heated at 60° C. for 30 min then cooled to room temperature.

Celite (125 g; 5 Wt % of theory yield) was added to the reaction mixture. The reaction was filtered and flask is rinsed with 1-2 L of toluene. The cake was then washed with 1-2 L of toluene.

The filtrate was transferred into a 100 L cylindrical extractor and warmed to 55° C. Water (10 L) was added slowly so as to maintain the temperature. The mixture was stirred for 30 minutes, then the layers were separated.

The organic layer was transferred to a 50 L round bottom flask and concentrated to a volume of 12 L or less. To the resulting mixture was added EtOAc (12 L). Stirred for at least two hours or overnight.

The resulting solids were filtered and the cake washed with a 1:1 mixture of EtOAc/Toluene (1.3 L). The solids were then dried.

### Example 4

Deprotection of 3-2

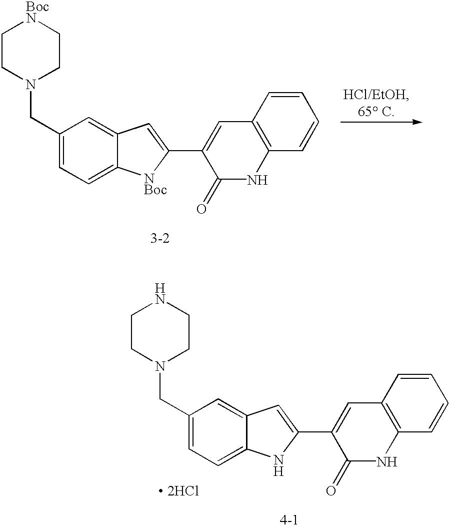

A slurry of quinindole 3-2, prepared as described in Example 3, (1.85 Kg) in absolute ethanol (28 L) was treated with concentrated aq HCl (3.7 L) in a 50 L flask. The solution was heated to 65° C. for 8 hours or more, then cooled to room temperature. The secondary amine as the bis-HCl salt was collected by filtration, with a 5 L ethanol wash.

### Example 5

Methylsulfonation of Intermediate 4-1

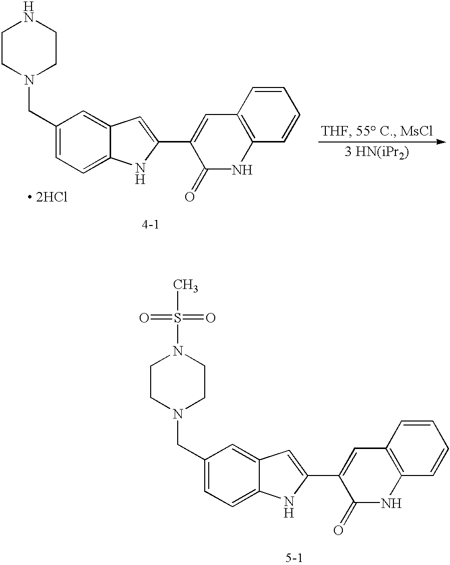

Intermediate 4-1 (1.2 kg, 2.78 moles), THF (24 L) and diisopropylamine (1.17 L, 8.35 moles) were charged to a 50 L round bottomed flask, and the slurry was heated to 55° C. Methanesulfonyl chloride was added over 3 hours, and the thick yellow slurry was stirred 4 hours or overnight. The mixture was cooled to room temperature, then water (15.6 L) was charged over 1.5 hours. To the last five liters of water was added 600 mL conc ammonium hydroxide to adjust the slurry pH to >7. (Total volume 43 L.) The slurry was aged one hour, then filtered, with a 3.6 L cake wash (60:40 THF: water). The final product was dried at 70° C. at 40 torr for several days, to provide compound 5-1 as a yellow solid.

Alternatively, the reaction can be quenched with 24 L total water.

Biological Assays

The compounds prepared by the process of the instant invention described in the Examples may be tested by the assays described below to assess kinase inhibitory activity. Other assays are known in the literature and could be readily performed by those of skill in the art. (See, for example, Dhanabal et al., Cancer Res. 59:189-197; Xin et al., J. Biol. Chem. 274:9116-9121; Sheu et al., Anticancer Res. 18:4435-4441; Ausprunk et al., Dev. Biol. 38:237-248; Gimbrone et al., J. Natl. Cancer Inst. 52:413-427; Nicosia et al., In Vitro 18:538-549.)

I. VEGF Receptor Kinase Assay

VEGF receptor kinase activity is measured by incorporation of radio-labeled phosphate into polyglutamic acid, tyrosine, 4:1 (pEY) substrate. The phosphorylated pEY product is trapped onto a filter membrane and the incorporation of radio-labeled phosphate quantified by scintillation counting.

Materials

VEGF Receptor Kinase

The intracellular tyrosine kinase domains of human KDR (Terman, B. I. et al. Oncogene (1991) vol. 6, pp. 1677-1683.) and Flt-1 (Shibuya, M. et al. Oncogene (1990) vol. 5, pp. 519-524) were cloned as glutathione S-transferase (GST) gene fusion proteins. This was accomplished by cloning the cytoplasmic domain of the KDR kinase as an in frame fusion at the carboxy terminus of the GST gene. Soluble recombinant GST-kinase domain fusion proteins were expressed in Spodoptera frugiperda (Sf21) insect cells (Invitrogen) using a baculovirus expression vector (pAcG2T, Pharmingen).

The other materials used and their compositions were as follows:

Lysis buffer: 50 mM Tris pH 7.4, 0.5 M NaCl, 5 mM DTT, 1 mM EDTA, 0.5% triton X-100, 10% glycerol, 10 mg/mL of each leupeptin, pepstatin and aprotinin and 1 mM phenylmethylsulfonyl fluoride (all Sigma).

Wash buffer: 50 mM Tris pH 7.4, 0.5 M NaCl, 5 mM DTT, 1 mM EDTA, 0.05% triton X-100, 10% glycerol, 10 mg/mL of each leupeptin, pepstatin and aprotinin and 1 mM phenylmethylsulfonyl fluoride.

Dialysis buffer: 50 mM Tris pH 7.4, 0.5 M NaCl, 5 mM DTT, 1 mM EDTA, 0.05% triton X-100, 50% glycerol, 10 mg/mL of each leupeptin, pepstatin and aprotinin and 1 mM phenylmethylsuflonyl fluoride.

10×reaction buffer: 200 mM Tris, pH 7.4, 1.0 M NaCl, 50 mM MnCl₂, 10 mM DTT and 5 mg/mL bovine serum albumin (Sigma).

Enzyme dilution buffer: 50 mM Tris, pH 7.4, 0.1 M NaCl, 1 mM DTT, 10% glycerol, 100 mg/mL BSA.

10×Substrate: 750 μg/mL poly (glutamic acid, tyrosine; 4:1) (Sigma).

Stop solution: 30% trichloroacetic acid, 0.2 M sodium pyrophosphate (both Fisher).

Wash solution: 15% trichloroacetic acid, 0.2 M sodium pyrophosphate.

Filter plates: Millipore #MAFC NOB, GF/C glass fiber 96 well plate.

Method

A. Protein Purification

1. Sf21 cells were infected with recombinant virus at a multiplicity of infection of 5 virus particles/ cell and grown at 27° C. for 48 hours.

2. All steps were performed at 4° C. Infected cells were harvested by centrifugation at 1000×g and lysed at 4° C. for 30 minutes with 1/10 volume of lysis buffer followed by centrifugation at 100,000×g for 1 hour. The supernatant was then passed over a glutathione Sepharose column (Pharmacia) equilibrated in lysis buffer and washed with 5 volumes of the same buffer followed by 5 volumes of wash buffer. Recombinant GST-KDR protein was eluted with wash buffer/10 mM reduced glutathione (Sigma) and dialyzed against dialysis buffer.

B. VEGF Receptor Kinase Assay

1. Add 5 μl of inhibitor or control to the assay in 50% DMSO.

2. Add 35 μl of reaction mix containing 5 μl of 10×reaction buffer, 5 μl 25 mM ATP/10 μCi [³³P]ATP (Amersham), and 5 μl 10×substrate.

3. Start the reaction by the addition of 10 μl of KDR (25 nM) in enzyme dilution buffer.

4. Mix and incubate at room temperature for 15 minutes.

5. Stop by the addition of 50 μl stop solution.

6. Incubate for 15 minutes at 4° C.

7. Transfer a 90 μl aliquot to filter plate.

8. Aspirate and wash 3 times with wash solution.

9. Add 30 μl of scintillation cocktail, seal plate and count in a Wallac Microbeta scintillation counter.

II. Human Umbilical Vein Endothelial Cell Mitogenesis Assay

Human umbilical vein endothelial cells (HUVECs) in culture proliferate in response to VEGF treatment and can be used as an assay system to quantify the effects of KDR kinase inhibitors on VEGF stimulation. In the assay described, quiescent HUVEC monolayers are treated with vehicle or test compound 2 hours prior to addition of VEGF or basic fibroblast growth factor (bFGF). The mitogenic response to VEGF or bFGF is determined by measuring the incorporation of [³H]thymidine into cellular DNA.

Materials

HUVECs: HUVECs frozen as primary culture isolates are obtained from Clonetics Corp. Cells are maintained in Endothelial Growth Medium (EGM; Clonetics) and are used for mitogenic assays described in passages 3-7 below.

Culture Plates: NUNCLON 96-well polystyrene tissue culture plates (NUNC #167008).

Assay Medium: Dulbecco's modification of Eagle's medium containing 1 g/mL glucose (low-glucose DMEM; Mediatech) plus 10% (v/v) fetal bovine serum (Clonetics).

Test Compounds: Working stocks of test compounds are diluted serially in 100% dimethylsulfoxide (DMSO) to 400-fold greater than their desired final concentrations. Final dilutions to 1×concentration are made directly into Assay Medium immediately prior to addition to cells.

10×Growth Factors: Solutions of human VEGF₁₆₅ (500 ng/mL; R&D Systems) and bFGF (10 ng/mL; R&D Systems) are prepared in Assay Medium.

10×[³H]Thymidine: [Methyl-³H]thymidine (20 Ci/mmol; Dupont-NEN) is diluted to 80 μCi/mL in low-glucose DMEM.

Cell Wash Medium: Hank's balanced salt solution (Mediatech) containing 1 mg/mL bovine serum albumin (Boehringer-Mannheim).

Cell Lysis Solution: 1 N NaOH, 2% (w/v) Na₂CO₃.

Method

1. HUVEC monolayers maintained in EGM are harvested by trypsinization and plated at a density of 4000 cells per 100 μL Assay Medium per well in 96-well plates. Cells are growth-arrested for 24 hours at 37° C. in a humidified atmosphere containing 5% CO₂.

2. Growth-arrest medium is replaced by 100 μL Assay Medium containing either vehicle (0.25% [v/v] DMSO) or the desired final concentration of test compound. All determinations are performed in triplicate. Cells are then incubated at 37° C. with 5% CO₂ for 2 hours to allow test compounds to enter cells.

3. After the 2-hour pretreatment period, cells are stimulated by addition of 10 μL/well of either Assay Medium, 10×VEGF solution or 10×bFGF solution. Cells are then incubated at 37° C. and 5% CO₂.

4. After 24 hours in the presence of growth factors, 10×[³H]thymidine (10 μL/well) is added.

5. Three days after addition of [³H]thymidine, medium is removed by aspiration, and cells are washed twice with Cell Wash Medium (400 μL/well followed by 200 μL/well). The washed, adherent cells are then solubilized by addition of Cell Lysis Solution (100 μL/well) and warming to 37° C. for 30 minutes. Cell lysates are transferred to 7-mL glass scintillation vials containing 150 μL of water. Scintillation cocktail (5 mL/vial) is added, and cell-associated radioactivity is determined by liquid scintillation spectroscopy.

What is claimed is:

1. A process for the preparation of a compound of Formula I, 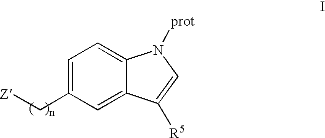
or a salt or optical isomer thereof;
wherein,

| | |
|---|---|
| a is | 0 or 1; |
| b is | 0 or 1; |
| m is | 0, 1, or 2; |
| n is | 1 to 10; |

Z′ is —NR⁷R⁸ or a protected precursor to —NR⁷R⁸;
R⁵ is selected from:
1) H,
2) (C═O)ₐO_{b}C₁-C₁₀ alkyl,
3) (C═O)ₐO_{b}aryl,
4) (C═O)ₐO_{b}C₂-C₁₀ alkenyl,
5) (C═O)ₐO_{b}C₂-C₁₀ alkynyl,
6) CO₂H,
7) halo,
8) OH,
9) O_{b}C₁-C₆ perfluoroalkyl,
10) (C═O)ₐNR⁷R⁸,
11) CN,
12) (C═O)ₐO_{b}C₃-C₈ cycloalkyl, and
13) (C═O)ₐO_{b}heterocyclyl,
said alkyl, aryl, alkenyl, alkynyl, cycloalkyl, and heterocyclyl is optionally substituted with one or more substituents selected from R⁶;
R⁶ is:
1) (C═O)ₐO_{b}C₁-C₁₀ alkyl,
2) (C═O)ₐO_{b}aryl,
3) C₂-C₁₀ alkenyl,
4) C₂-C₁₀ alkynyl,
5) (C═O)ₐO_{b} heterocyclyl,
6) CO₂H,
7) halo,
8) CN,
9) OH,
10) O_{b}C₁-C₆ perfluoroalkyl,
11) Oₐ(C═O)_{b}NR⁷R⁸,
12) oxo,
13) CHO,
14) (N═O)R⁷R⁸, or
15) (C═O)ₐO_{b}C₃-C₈ cycloalkyl,
said alkyl, aryl, alkenyl, alkynyl, heterocyclyl, and cycloalkyl optionally substituted with one or more substituents selcted from R^{6a};
R^{6a} is selected from:
1) (C═O)ᵣOₛ(C₁-C₁₀)alkyl, wherein r and s are independently 0 or 1,
2) Oᵣ(C₁-C₃)perfluoroalkyl, wherein r is 0 or 1,
3) (C₀-C₆)alkylene-S(O)ₘR^{a}, wherein m is 0, 1, or 2,
4) oxo,
5) OH,
6) halo,
7) CN,
8) (C₂-C₁₀)alkenyl,
9) (C₂-C₁₀)alkynyl,
10) (C₃-C₆)cycloalkyl,
11) (C₀-C₆)alkylene-aryl,
12) (C₀-C₆)alkylene-heterocyclyl,
13) (C₀-C₆)alkylene-N(R^{b})₂,
14) C(O)R^{a},
15) (C₀-C₆)alkylene-CO₂R^{a},
16) C(O)H, and
17) (C₀-C₆)alkylene-CO₂H,
said alkyl, alkenyl, alkynyl, cycloalkyl, aryl, and heterocyclyl is optionally substituted with up to three substituents selected from R^{b}, OH, (C₁-C₆)alkoxy, halogen, CO₂H, CN, O(C═O)C₁-C₆ alkyl, oxo, and N(R^{b})₂;
R⁷ and R⁸ are independently selected from:
1) H,
2) (C═O)O_{b}C₁-C₁₀ alkyl,
3) (C═O)O_{b}C₃-C₈ cycloalkyl,
4) (C═O)O_{b}aryl,
5) (C═O)O_{b}heterocyclyl,
6) C₁-C₁₀ alkyl,
7) aryl,
8) C₂-C₁₀ alkenyl,
9) C₂-C₁₀ alkynyl,
10) heterocyclyl,
11) C₃-C₈ cycloalkyl,
12) SO₂R^{a}, and
13) (C═O)NR^{b}₂,
said alkyl, cycloalkyl, aryl, heterocylyl, alkenyl, and alkynyl is optionally substituted with one or more substituents selected from R6^{a}, or
R⁷ and R⁸ can be taken together with the nitrogen to which they are attached to form a monocyclic or bicyclic heterocycle with 5-7 members in each ring and optionally containing, in addition to the nitrogen, one or two additional heteroatoms selected from N, O and S, said monocylcic or bicyclic heterocycle optionally substituted with one or more substituents selected from R^{6a};
R^{a} is (C₁-C₆)alkyl, (C₃-C₆)cycloalkyl, aryl, or heterocyclyl;
R^{b} is H, (C₁-C₆)alkyl, aryl, heterocyclyl, (C₃-C₆)cycloalkyl, (C═O)OC₁-C₆ alkyl, (C═O)C₁-C₆ alkyl or S(O)₂R^{a}; and
prot is a nitrogen protecting group;
which comprises the steps of
a) reacting the compound of the formula II: 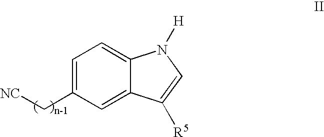
wherein n and R⁵ are defined hereinabove,
with a nitrogen protecting group reagent to provide a compound of the formula III: 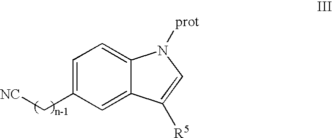
wherein n and R⁵ are defined hereinabove;
b) reacting the compound of the formula III with a cyano reducing agent to provide a compound of the formula IV: 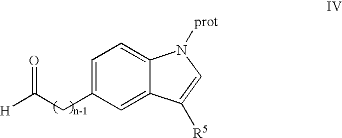
wherein n and R⁵ are defined hereinabove; and
c) reacting the intermediate of the formula IV with an amine HZ′,
wherein Z′ is as defined hereinabove, in the presence of a reducing agent to provide the compound of the formula I.

2. The process according to claim 1, wherein the nitrogen protecting group in step a) is FMOC, CBZ or Boc.

3. The process according to claim 1, wherein the cyano substituent of the protected cyano-indole in step b) is reduced with a cyano reducing agent selected from DiBAL-H, LiAlH₄, NaAlH₄ or LiAlH(OEt)₃.

4. The process according to claim 1, wherein the reducing agent in step c) is selected from H₂ with Pd/C, H₂ with PtO₂, sodium borohydride, sodium cyanoborohydride, BH₃/pyridine and sodium triacetoxyborohydride.

5. The process according to claim 1, wherein amine HZ′ is selected from: 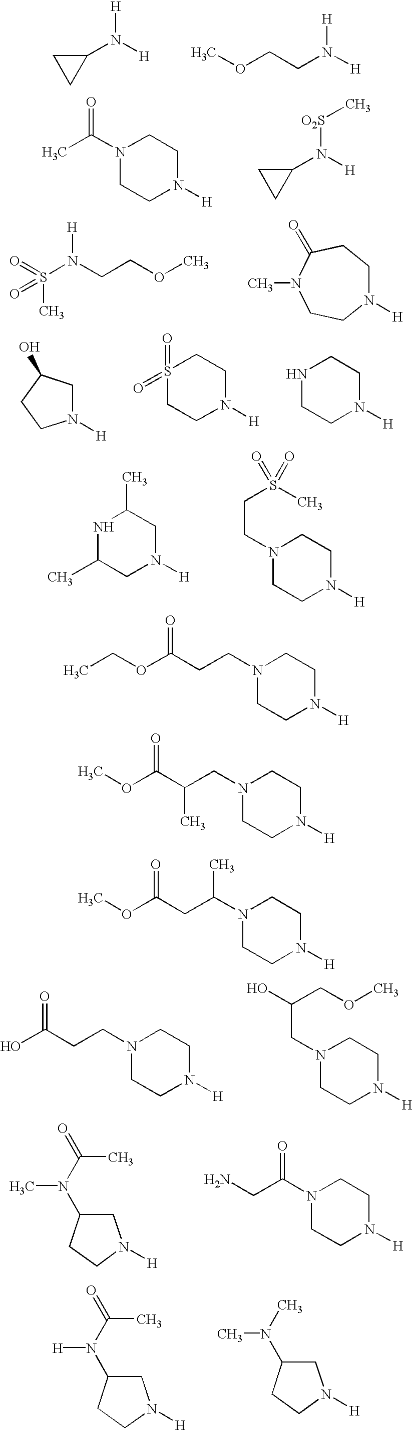
or a protected precusor to one of the amines above or a salt thereof.

6. A process for the preparation of a compound of Formula Ia: 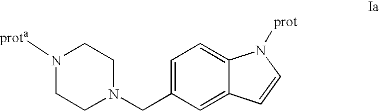
or a salt thereof;
wherein, prot and prot^{a} are independently selected from a nitrogen protecting group;
which comprises the steps of
e) reacting the compound of the formula IIa: 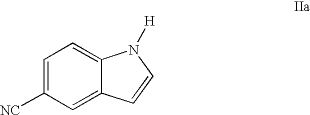
with a nitrogen protecting group reagent to provide a compound of the formula IIIa: 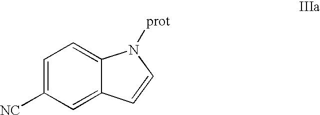
wherein prot is defined hereinabove;
f) reacting the compound of the formula IIIa with a cyano reducing agent to provide a compound of the formula IVa: 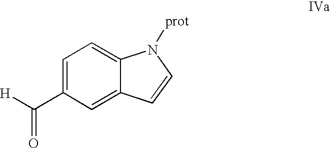
wherein prot is defined hereinabove; and
d) reacting the intermediate of the formula IVa with the amine 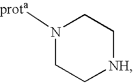
wherein prot^{a} is as defined hereinabove in the presence of a reducing agent to provide the compound of the formula Ia.

7. A process for the preparation of a compound of Formula 1-4: 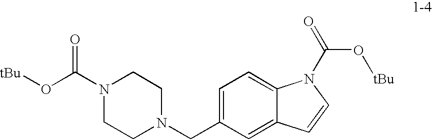
or a salt thereof;
that comprises the steps of
a) reacting the compound of the formula IIa: 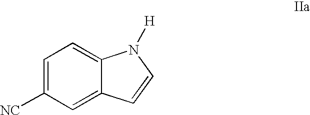
with a t-butoxycarbonyl anhydride to provide a compound of the formula IIIb: 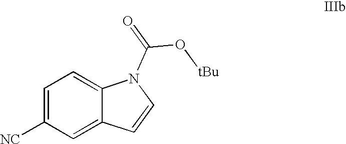
b) reacting the compound of the formula IIIb with a cyano reducing agent to provide a compound of the formula 1-2: 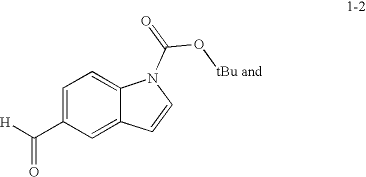
c) reacting the intermediate of the formula 1-2 with N-t-butoxycarbonylpiperidine in the presence of a reducing agent to provide the compound of the formula 1-4.

8. The process according to claim 7 wherein the protected cyano-indole of Formula IIIb in step a) is formed by reacting the cyano-indole of Formula IIa with di-tert-butyl dicarbonate and a catalytic amount of 4-dimethylaminopyridine at a temperature of about 20° C. to about 30° C.; the reduction in step b) is accomplished by using diisobutylaluminum hydride to form an aldehyde-substituted indole of Formula 1-2; 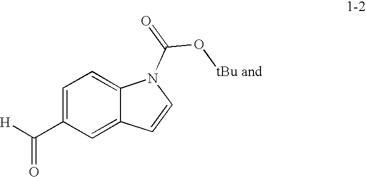
(c) reacting the aldehyde-substituted indole from step (b) with 4-tert-(butoxycarbonyl)piperazine in the presence of sodium triacetoxyborohydride to form the compound of Formula 1-4.

9. A compound of the formula 1-4 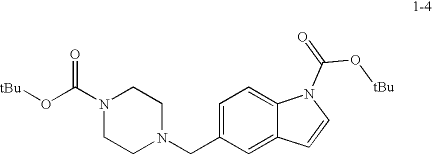
or a salt thereof.
